(19) United States
(12) Patent Application Publication
(10) Pub. No.: US 2023/0147489 A1
(43) Pub. Date: May 11, 2023

(54) SYSTEMS AND METHODS FOR PHYSIOLOGY MONITORING GARMENT

(71) Applicant: Myant Inc., Etobicoke (CA)

(72) Inventors: Tony Chahine, Toronto (CA); Michelle Zheng, Toronto (CA); Parth Satvik Jain, Toronto (CA); Patricia Pamela Lim, Toronto (CA); Abdul Qadir Javaid, Toronta (CA); John Persic, Toronto (CA)

(21) Appl. No.: 18/081,972

(22) Filed: Dec. 15, 2022

Related U.S. Application Data
(63) Continuation of application No. 17/421,088, filed on Jul. 7, 2021.
(60) Provisional application No. 62/789,361, filed on Jan. 7, 2019.

(51) Int. Cl. A61B 5/00; A61B 5/021; A61B 5/024; A61B 5/053; A61B 5/11

(52) U.S. Cl. CPC A61B 5/6804; A61B 5/053; A61B 5/1102; A61B 5/02125; A61B 5/02416

(57) ABSTRACT

A garment for detecting physiological data. The garment may include a garment body and a primary sensor panel affixed to a user facing side of the garment body. The primary sensor panel may include at least one bio signal sensor type to generate a primary set of bio signals. The garment may include a processor coupled to the primary sensor panel and a memory coupled to the processor. The memory may store processor-executable instructions that, when executed, configure the processor to: receive, from the primary sensor panel, the primary set of bio signals; generate a bio signal waveform based on the primary set of bio signals; and determine a hemodynamic metric associated with the user based on the bio signal waveforms associated with the user.

### CROSS REFERENCE TO RELATED APPLICATIONS

This application claims priority from U.S. provisional patent application no. 62/789,361, filed on Jan. 7, 2019, the entire contents of which are hereby incorporated by reference herein.

### FIELD

Embodiments of the present disclosure generally relate to the field of smart garments, and in particular to garments for detecting physiological data.

### BACKGROUND

Specialized apparatus or devices for measuring physiological data, such as blood pressure, may be secured to a patient user during physiological data acquisition. For example, a sphygmomanometer in combination with a stethoscope may be configured to determine blood pressure of a patient user. The sphygmomanometer may include an inflatable cuff to collapse and subsequently release a patient user’s artery in a controlled manner for determining blood pressure of the patient user. Such specialized equipment may be intended to be worn by a user for a short duration of time.

### SUMMARY

The present application describes smart garments for monitoring physiological conditions, such as blood pressure or other physiological metrics, of garment users. The garment may be disposed on a portion of the user’s body and may include one or more bio signal sensors affixed to a user facing side of the garment. The garment may be configured to position and/or retain the one or more bio signal sensors against the user limb with substantially consistent pressure to continuously detect or generate bio signals over time for physiological monitoring of the garment user. In some examples, the one or more bio signal sensors may include at least two bio signal sensor types, and physiological metric may be determined based on a combination of bio signal waveform data associated with each of the at least two bio signal sensor types.

In one aspect, the present application provides a garment for detecting physiological data. The garment may include a garment body and a primary sensor panel affixed to a user facing side of the garment body. The primary sensor panel may include at least one bio signal sensor type to generate a primary set of bio signals. The garment may include a processor coupled to the primary sensor panel and a memory coupled to the processor. The memory may store processor-executable instructions that, when executed, configure the processor to: receive, from the primary sensor panel, the primary set of bio signals; generate a bio signal waveform based on the primary set of bio signals; and determine a hemodynamic metric associated with the user based on the bio signal waveforms associated with the user.

In some embodiments, the bio signal waveform may be based on pulse transit time data. Determining the hemodynamic metric may include determining a blood pressure measure based on the pulse transit time data.

In some embodiments, the garment may include at least one of an accelerometer or a piezo sensor integrated in the garment body. Receiving the primary set of bio signals may be in response to receiving a trigger signal generated by at least one of the accelerometer or the piezo sensor indicating movement of the user.

In some embodiments, the primary sensor panel includes at least two bio signal sensor types. Determining the hemodynamic metric may be based on a combination of bio signal waveform data associated with each of the at least two bio signal sensor types.

In some embodiments, the primary sensor panel may include at least one of a photoplethysmogram (PPG) sensor, an electrocardiogram (ECG) sensor, or a ballistocardiogram (BCG) sensor.

In some embodiments, the primary sensor panel may include a pair of electrical bio impedance sensors measuring electrical blood conductivity for determining the hemodynamic metric.

In some embodiments, the garment may include a complementary sensor panel distal from the primary sensor panel and affixed to the user limb facing side of the garment body. The complementary sensor panel may be configured to generate a secondary set of bio signals.

In some embodiments, the garment may include a conductive fibre knitted in the garment body and configured to conduct at least one of a data signal or a power signal. The conductive fibre may interconnect the primary sensor panel and the complementary sensor panel.

In some embodiments, the primary set of bio signals and the secondary set of bio signals may be a differential set of bio signals. Determining the hemodynamic metric associated with the user may be based on the differential set of bio signals.

In another aspect, the present application provides a garment for detecting physiological data. The garment may include a garment body and a primary sensor panel affixed to a user facing side of the garment body. The primary sensor panel may include at least one bio signal sensor to generate a primary set of bio signals for determining hemodynamic data associated with a user. The garment body may include a garment band coupled to the sensor panel to retain the sensor panel against the user limb with substantially consistent pressure.

In some embodiments, the primary sensor panel may be configured to generate pulse transit time data for determining a blood pressure metric associated with the user.

In some embodiments, the primary sensor panel may include a pair of electrical bio impedance sensors measuring electrical blood conductivity for determining the hemodynamic data.

In some embodiments, the garment is a shirt configured to be worn on an upper body of the user. The primary sensor panel may be positioned on a shirt sleeve.

In some embodiments, the garment may include a complementary sensor panel distal from the primary sensor panel and affixed to the user limb facing side of the garment body. The complementary sensor panel may be configured to generate a secondary set of bio signals.

In some embodiments, the garment may include a conductive fibre knitted in the garment body and configured to conduct at least one of a data signal or a power signal. The conductive fibre may interconnect the primary sensor panel and the complementary sensor panel.

In some embodiments, the conductive fibre may be knitted into a garment seam of the garment.

In some embodiments, the primary sensor panel may include at least one of a photoplethysmogram (PPG) sensor, an electrocardiogram (ECG) sensor, or a ballistocardiogram (BCG) sensor.

In some embodiments, the garment may include a textile enclosure defining a cavity and projecting from the garment body. The textile enclosure may be configured to electrically interconnect the primary sensor panel and a controller device receivable by the textile enclosure.

In some embodiments, the garment may include at least one of an accelerometer or a piezo sensor coupled to the primary sensor panel to generate a trigger signal, in response to detected user movement, to trigger generation of the primary set of bio signals.

In another aspect, a non-transitory computer-readable medium or media having stored thereon machine interpretable instructions which, when executed by a processor may cause the processor to perform one or more methods described herein.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a system for detecting physiological data, in accordance with an embodiment of the present application;

FIG. 2 illustrates a front view of a garment for detecting physiological data, in accordance with an embodiment of the present application;

FIG. 3 illustrates a rear view of the garment of FIG. 2;

FIG. 4 illustrates a side view of the garment of FIG. 2;

FIG. 5 illustrates an elevation view of a garment, in accordance with another embodiment of the present application;

FIGS. 6A and 6B illustrate a front perspective view and a rear perspective view, respectively, of a garment for detecting physiological data, in accordance with an embodiment of the present application;

FIG. 7 illustrates a garment sleeve, in accordance with an embodiment of the present application;

FIGS. 8A and 8B illustrate plan views of shirt yokes, in accordance with embodiments of the present application;

FIG. 9 illustrates a flowchart of a method of monitoring physiological conditions, in accordance with an embodiment of the present application; and

FIG. 10 illustrates a block diagram of a computing device, in accordance with an embodiment of the present application.

### DETAILED DESCRIPTION

Specialized devices may be configured for determining physiological metrics of a user. For example, a combination of a sphygmomanometer and a stethoscope may be used for determining a user’s blood pressure. The sphygmomanometer may include an inflatable cuff for collapsing a user’s artery and, subsequently, releasing the user’s artery in a controlled manner for determining blood pressure of the patient user. Upon collapsing and releasing the patient user’s artery, the stethoscope may be used to determine at what pressure blood begins flowing in the artery, and at what pressure the blood flow becomes unimpeded. Such specialized equipment and methods for measuring blood pressure may be intended to be worn by a user for a short time duration, and may not be intended to be worn for extended periods of time. Such specialized equipment and methods may not be suitable for hemodynamic monitoring over an extended period of time. Further, such specialized equipment may be invasive or uncomfortable to the user. The user may experience discomfort as the inflatable cuff may be used to collapse an artery, preventing blood flow. Less invasive devices for physiological monitoring (e.g., hemodynamic monitoring, etc.) may be desirable.

In some embodiments of the present application, devices or apparatus for physiological monitoring, such as hemodynamic or blood pressure monitoring, may be provided in a garment. The garment may be a t-shirt or a long sleeve shirt having one or more sleeves for receiving a patient user’s arms. At least one shirt sleeve may include a sensor array configured to be secured with substantially consistent pressure to the patient user’s arm. Because example garments described in the present application may generate and store physiological data over time, in some scenarios, trends and deviations therefrom may be determined.

Examples described in the present application may be directed to hemodynamic monitoring, such as blood pressure monitoring, based on physiological data acquisitions using a sensor array that may be secured to a user limb. It may be appreciated that devices for measuring other physiological metrics based on one or more sensor arrays secured, via consistent pressure, to any other type of user limb or body part may be contemplated. Embodiments described in the present application may be directed to shirts and shirt sleeves. It may be appreciated that the apparatus and devices for acquiring physiological data may be provided for other types of garments, such as pants, hats, or other types of garments that may receive a user limb or a part of the user’s body.

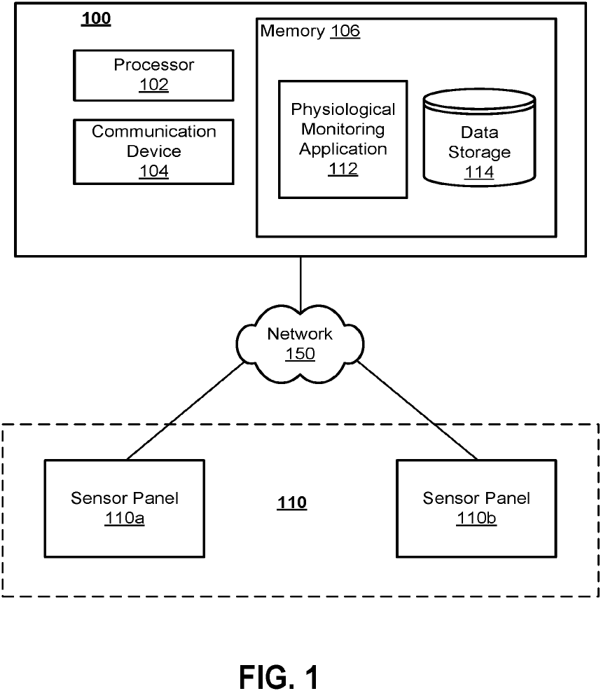

Reference is made to FIG. 1, which illustrates a system for detecting physiological data, in accordance with an embodiment of the present application. The system may include a controller device 100 and one or more sensor panels 110.

In some embodiments, the one or more sensor panels 110 may be affixed to a garment, and the one or more sensor panels 110 may be positioned proximal to or against a user’s skin for detecting physiological data. In some embodiments, the one or more sensor panels 110 may include at least one bio signal sensor positioned on a user limb facing side of the garment. In some embodiments, the one or more sensor panels 110 may generate bio signals. The controller device 100 may receive the generated bio signals and may conduct operations for determining physiological data associated with the user.

In some embodiments, the controller device 100 may be a computing device for transmitting or receiving data messages to or from the one or more sensor panels 110.

The controller device 100 may be coupled to the at least one sensor panels 110 via a network 150. The network 150 may include any wired or wireless communication path, such as an electrical circuit. In some embodiments, the network 150 may include one or more busses, interconnects, wires, circuits, and/or any other connection and/or control circuit, or a combination thereof. In some embodiments, the network 150 may include a wired or a wireless wide area network (WAN), local area network (LAN), a combination thereof, or the like. In some embodiments, the network 150 may include a Bluetooth® network, a Bluetooth® low energy network, a short-range communication network, or the like. The network 150 may be a communication interface such that the controller device 100 and the at least one sensor panel 110 may communicate.

In some embodiments, the system illustrated in FIG. 1 may be integrated into a garment, such as a t-shirt, a long sleeve shirt, or other type of garment that may be worn by a user. For example, a t-shirt may be an athletic shirt. In the example of FIG. 1, the sensor panels 110 may include a first sensor panel 110a and a second sensor panel 110b. The first sensor panel 110a may be affixed to a portion of a first shirt sleeve on a user facing side such that, when a user wears the garment, the first sensor panel 110a may be configured to be proximal to or contact the user’s arm.

The second sensor panel 110b may be affixed to a portion of a second shirt sleeve on a user facing side such that, when a user wears the garment, the second sensor panel 110b may be configured to be proximal to or contact the user’s arm. The first sensor panel 110a and the second sensor panel 110b may be electrically interconnected by a conductive fibre that may be knitted into the garment. In some embodiments, bio signal data associated with the first sensor panel 110a and the second sensor panel 110b may, in combination, be differential bio signals, such that bio signal noise that otherwise would be present with single-ended signals may be reduced during bio signal processing.

Although two sensor panels 110 are illustrated in FIG. 1, any number of sensor panels 110 may be contemplated. In some embodiments, one or more of the sensor panels 110 may be affixed to a cuff of a shirt sleeve, and when a user limb is received within the cuff of the shirt sleeve, one or more of the sensor panels 110 may be positioned for contacting the user’s arm.

In some embodiments, the controller device 100 may be integrated into the garment and may be coupled to the sensor panels 110 via electrical interconnection means, such as via one or more electrical circuits. In some embodiments, the controller device 100 may be removably mounted to the garment, such that the controller device 110 may be removed when the garment is cleaned or laundered. In some embodiments, the garment may include a pocket-like textile enclosure projecting from the garment body. The pocket-like textile enclosure may define a cavity configured to receive the controller device 100. The pocket-like textile enclosure may include features to electrically interconnect the controller 100 and the one or more sensor panels 110. In some embodiments, the pocket-like textile enclosure may include textile material substantially similar to textile material of the garment body. In some embodiments, the pocket-like textile enclosure may include textile material having moisture resistant properties, such that the pocket-like textile enclosure may provide a moisture barrier to the controller device 100.

The controller device 100 may receive one or more physiological data sets from the one or more sensor panels 110 and may conduct operations for analyzing the one or more physiological data sets for determining physiological metrics, such as blood pressure. In some embodiments, the controller device 100 may be configured to determine other physiological metrics, such as heart rate data, respiratory data, olfactory data, or other types of physiological data. In some embodiments, the controller device 100 may conduct operations for estimating physiological metrics associated with the user, including heart rate data, arrhythmias such as atrial fibrillation, blood pressure, user steps / movement, calorie count, user activity, user sleep quality, user sleep related breathing characteristics, or other physiological metrics.

In some embodiments, the garment may be a smart garment formed of a knitted textile. In some embodiments, the garment may be formed of other textile forms and/or techniques such as weaving, knitting (warp, weft, etc.) or the like. In some embodiments, the smart garment may include one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. Pat. Application No. 15/267,818, the entire contents of which are herein incorporated by reference.

As used herein, “textile” may refer to material made or formed by manipulating natural or artificial fibres to interlace or to create an organized network of fibres. Textiles may be formed using yarn, where yarn refers to a long continuous length of a plurality of fibres that may be interlocked (i.e., fitting into each other, as if twined together, or twisted together). Herein, the terms fibre and yarn may be used interchangeably. Fibres or yarns can be manipulated to form a textile according to example methods that provide an interlaced organized network of fibres, including but not limited to weaving, knitting, sew and cut, crocheting, knotting and felting.

Various sections of a textile may be integrally formed into a layer to utilize different structural properties of different types of fibres. For example, conductive fibres may be manipulated to form networks of conductive fibres. Non-conductive fibres may be manipulated to form networks of non-conductive fibers. The networks of fibres may include different sections of a textile by integrating the networks of fibres into a layer of the textile. The networks of conductive fibres may form one or more conductive pathways that may electrically connect sensors and actuators embedded in the smart garment, for conveying data and/or power to and/or from the respective aforementioned devices.

In some embodiments, the sensors embedded in the smart garment may be the one or more sensor panels 110 for detecting physiological data. The network 150 may include the network of conductive fibres of the smart textile for conveying data and/or power between the one or more sensor panels 110 and the controller device 100. The network 150 may include at least one conductive fibre configured as a conductive pathway.

In some embodiments, the at least one conductive fibres may be knitted into the garment. In some embodiments, the at least one conductive fibres may be knitted into a garment seam. In some embodiments, the conductive fibers may be geometrically jointed or configured to reduce or suppress signal noise when power or signals may be transmitted along the conductive fibres knitted into the garment seams.

In some embodiments, multiple layers of textile may be stacked upon each other to provide a multi-layer textile.

In the present application, “interlace” may refer to fibres (either artificial or natural) crossing over and/or under one another in an organized fashion, typically alternately over and under one another, in a layer. When interlaced, adjacent fibres may touch each other at intersection points (e.g., points where one fibre may cross over or under another fibre). In one example, first fibres extending in a first direction may be interlaced with second fibres extending laterally or transverse to the fibres extending in the first connection. In another example, the second fibres may extend laterally at 90 degrees from the first fibres when interlaced with the first fibres. Interlaced fibres extending in a sheet may be referred to as a network of fibres.

In the present application, “integrated” or “integrally” may refer to combining, coordinating or otherwise bringing together separate elements so as to provide a substantially harmonious, consistent, interrelated whole. In the context of a textile, the textile may have various sections comprising networks of fibres with different structural properties. For example, a textile may have a section comprising a network of conductive fibres and a section comprising a network of non-conductive fibres. Two or more sections comprising networks of fibres may be said to be “integrated” together into a textile (or “integrally formed”) when at least one fibre of one network is interlaced with at least one fibre of the other network such that the two networks form a layer of the textile. Further, when integrated, two sections of a textile may also be described as being substantially inseparable from the textile. Here, “substantially inseparable” refers to the notion that separation of the sections of the textile from each other results in disassembly or destruction of the textile itself.

In some examples, conductive fabric (e.g., group of conductive fibres) may be knit along with (e.g., to be integral with) the base fabric (e.g., surface) in a layer. Such knitting may be performed using a circular knit machine or a flat bed knit machine, or the like, from a vendor such as Santoni or Stoll.

The controller device 100 includes a processor 102 configured to conduct processor readable instructions that, when executed, configure the processor 102 to conduct operations described herein. The controller device 100 may include a communication device 104 to communicate with other computing or sensor devices, to access or connect to network resources, or to perform other computing applications by connecting to a network (or multiple networks) capable of carrying data. In some examples, the communication device 104 may include one or more busses, interconnects, wires, circuits, and/or any other connection and/or control circuit, or combination thereof. The communication device 104 may provide an interface for communicating data between the controller device 100 and the one or more sensor panels 110. In some embodiments, the one or more busses, interconnects, wires, circuits, or the like may be the network of conductive and non-conductive fibers of a smart textile.

The controller device 100 may include memory 106. The memory 106 may include one or a combination of computer memory, such as static random-access memory (SRAM), random-access memory (RAM), read-only memory (ROM), electro-optical memory, magneto-optical memory, erasable programmable read-only memory (EPROM), and electrically-erasable programmable read-only memory (EEPROM), Ferroelectric RAM (FRAM) or the like.

The memory 106 may store a physiological monitoring application 112 including processor readable instructions for conducting operations described herein. In some examples, the physiological monitoring application 112 may include operations for receiving and storing physiological data of a user. The physiological data of the user may include bio signal waveform data generated based on data received from the one or more sensor panels 110. The physiological monitoring application 112 may include operations to determine one or more physiological metric trends over time based on the physiological data (e.g., bio signal waveform data, or the like). In some embodiments, the physiological monitoring application 112 may include operations to conduct statistical analysis based on the physiological data for determining physiological metric trends. In some embodiments, statistical analysis may include operations to determine averages, mean, max/min, standard deviation measures, or other statistical measures of physiological metrics. By integrating the one or more sensor panels 110 into a garment, embodiments of the present application may be configured for a user to wear the garment for extended periods of time and for collecting physiological data with reduced discomfort. The one or more sensor panels 110 may be positioned against the user’s limb when the garment is worn by the user.

In some embodiments, the physiological monitoring application 112 may include operations for determining, based on the bio signal waveform data, physiological metrics, such as hemodynamic metrics associated with the user. In some embodiments, hemodynamic metrics may include blood pressure data. In some embodiments, the physiological metrics may include an estimation of user heart rate, identification of arrhythmias such as atrial fibrillation, blood pressure, user movement steps, calories burned, identification of user activity, identification of user sleep quality, identification of sleep related breathing characteristics, or the like.

The controller device 100 may include a data storage 114. In some embodiments, the data storage 114 may be a secure data store. In some embodiments, the data storage 114 may store received physiological data sets, such as blood pressure data, heart rate data, or other types of data. In some examples, the data storage 114 may store data associated with criteria for analyzing received physiological data sets. In some embodiments, the stored criteria may include blood pressure criteria that may be used for generating indications that blood pressure data may be trending beyond a defined blood pressure range. In some embodiments, the controller device 100 may be configured to monitor other types of physiological data or trends, and the stored criteria may include other physiological data criteria used for generating indications that physiological data may be trending beyond a defined metric range.

In some embodiments, the sensor panels 110 may include one or more sensors, and the one or more sensors may include one or a combination of electrocardiogram (ECG) sensors, photoplethysmogram (PPG) sensors, ballistocardiography (BCG) sensors, accelerometers, electro bio impedance sensors, or piezo sensors. Other types of sensors may be contemplated.

As embodiments of the garment may be worn by a user, the one or more sensor panels 110 may be positioned proximal to or may contact the user (e.g., user limb) for generating bio signals over time. In some embodiments, as the controller device 110 may configure the one or more sensor panels 110 to continuously detect or generate bio signals over time for monitoring a physiological condition of the user, the garment may be configured to continuously monitor physiological status of the user. Physiological status may include hemodynamic metrics (e.g., blood pressure metrics), or the like.

In some embodiments, the controller device 100 may be configured to periodically receive, from the one or more sensor panels 110, bio signals and may conduct operations for tracking abrupt changes in physiological status of the user. For example, when the controller device 100 conducts operations to monitor changes in the user’s blood pressure, the controller device 100 may identify a potentially adverse health event when the user’s blood pressure drops by more than a threshold amount within a determined period of time (e.g., rapid drop in blood pressure). When the controller 100 conducts operations to identify potentially adverse health events, the controller 100 may conduct operations to transmit alert signals to the user’s mobile device or to computing systems. In some embodiments, the controller 100 may conduct operations to activate one or more actuators embedded in the garment for providing feedback to the garment user. In some examples, potentially adverse health events may include fainting, confusion, heart attacks / strokes, dehydration, allergic reactions, shocks, hypothermic conditions, heat strokes, or other physical traumatic events. In some examples, the controller 100 may conduct operations to identify day-to-day movements of the user based on bio signals, such as a user abruptly standing up, etc.

In some embodiments, the controller device 100 may conduct operations to determine trending changes to the user over time, and the controller device 100 may conduct operations to infer that the user may be undergoing lifestyle changes, such as diet changes, health changes (e.g., organ function, aging), or the like.

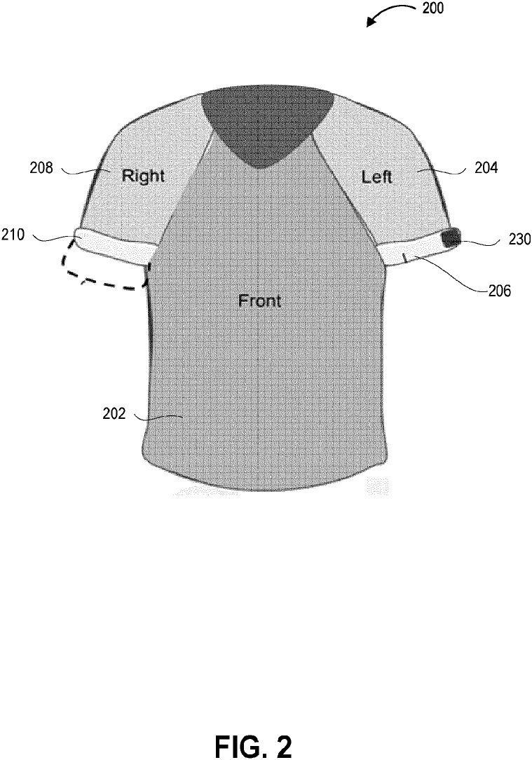

To obtain physiological sensor data readings in a repeatable way, the garment may include features to position the one or more sensor panels 110 against a user limb with substantially consistent pressure. In some embodiments, the fastening feature may be a garment band configured to retain the one or more sensor panels 110 against a user limb with substantially consistent pressure while the garment may be worn by a user. In some embodiments, from the experience or point of view of a user of embodiments of the present application, the garment may be configured to position / press the one or more sensor panels 110 against the user limb without any temporal tightening during data acquisition (e.g., without any tightening of the garment that is akin to a sphygmomanometer inflating to collapse a user’s artery during blood pressure measurements). That is, from the garment user’s point of view, the garment user may not experience any pressure on the user’s limb from the one or more sensor panels 110 or any tightening of the garment when the one or more sensor panels 110 detect or generate bio signals. In some embodiments, the one or more sensor panels 110 may be configured to generate bio signals based on physiological changes detected at the surface of the user’s limb. To illustrate embodiments of the present application, reference is made to FIG. 2.

FIG. 2 illustrates a front view of a garment 200 for detecting physiological data, in accordance with an embodiment of the present application. The garment 200 may be configured or adapted to be worn on an upper body of a user, and may be a long sleeve shirt, a t-shirt, a dress, or other type of upper body garment. In some embodiments, the garment 200 may be configured to be disposed over a lower body section of a garment user. The garment 200 may be a pair of pants, shorts, undergarment, or other type of garment. In some examples, the garment may be positioned to be proximal to legs of the garment user, such that garment bands or cuffs may wrap around angles, calves, thighs, or other lower body sections of the garment user.

The garment 200 may be configured to generate, based on a data sensor, one or more bio signals associated with a user limb or body part. In some embodiments, the garment 200 may be configured with one or more sensors, such as electrocardiogram (ECG) sensors, ballistocardiogram (BCG) sensors, electrical bio impedance sensors, or photoplethysmogram (PPG) sensors, to generate bio signals associated with a user. Generated bio signal data sets from each of a plurality of sensors may be used individually or in combination for determining cardiovascular parameters, hemodynamic parameters, or respiratory parameters, among other examples, associated with a user.

The garment 200 may include a front section 202, a first side section 204, and a second side section 208. In some embodiments, the first side section 204 may be associated with a left arm sleeve of the garment 200 and the second side section 208 may be associated with a right arm sleeve of the garment 200.

In some embodiments, the first side section 204 may include a first garment band 206 and the second side section 208 may include a second garment band 210. The first garment band 206 may be affixed and/or adjacent to the first side section 204 and the second garment band 210 may be affixed and/or adjacent to the second side section 208.

In some embodiments, the garment 200 may include a sensor panel 230. The sensor panel 230 may be coupled to the garment body on a user limb facing side of the garment 200. In some embodiments, the sensor panel 230 may be coupled to the first side section 204. In FIG. 2, the sensor panel 230 is illustrated as being coupled to the first side section 204. It may be appreciated that a further sensor panel (e.g., a complementary sensor panel) may be coupled to the second side section 208 or any other portion of the garment 200. The one or more sensor panels affixed to the garment 200 may be the one or more sensor panels 110 illustrated in FIG. 1.

In some embodiments, the garment 200 may include a first garment band 206 or a second garment band 210. In some embodiments, the first garment band 206 may be coupled to the sensor panel 230. The first garment band 206 may be configured to retain the sensor panel against the user limb with substantially consistent pressure. In FIG. 2, the second garment band 210 may be configured to retain a complementary sensor panel (not illustrated in FIG. 2) against the user limb with substantially consistent pressure. In some embodiments, the first garment band 206 or the second garment band 210 may include an elastomeric band and/or a latch device.

In the illustration of FIG. 2, the garment 200 includes a sensor panel 230 associated with the first side section 204. It may be appreciated that the garment 200 may include any number of sensor panels coupled to other portions of the garment 200, such as the second side section 208, the front section 202, or the like.

In some embodiments, the first side section 204 or the second side section 208 may include a sleeve roll-up design. Accordingly, the one or more sensor panels may not be positioned at a sleeve cuff, but may be positioned at any part of the garment sleeve.

In some embodiments, the garment 200 may include a sensor panel affixed to the user facing side on the first side section 204 and the garment 200 may include a complementary sensor panel affixed to the user facing side on the second side section 208. The garment 200 may include a controller device (not illustrated in FIG. 2) configured to conduct operations of periodically receiving bio signal data from the pair of sensor panels and may identify differences in the respective set of received bio signals beyond a threshold value for identifying potentially faulty bio signal data (e.g., one of the sensor panels may be faulty and providing an erroneous reading) or a potentially adverse user condition (e.g., the user may be experiencing a stroke, adverse artery / vein function, where blood pressure detected at an artery on one side of the body is very different than blood pressure detected at an opposing artery on another side of the body).

Although FIG. 2 is directed to a garment having sensor panels on opposing garment sleeves, in some embodiments, the garment may include sensor panels on garment portions that may be associated with legs, ankles, wrists, calves, or other portions of the user body.

In some embodiments, the sensor panel may include pairs of bio signal sensors for generating differential signals. Accordingly, the controller device (not illustrated in FIG. 2) coupled to the garment 200 may receive differential bio signals, such that bio signal noise that otherwise may be present with single-ended signals may be reduced.

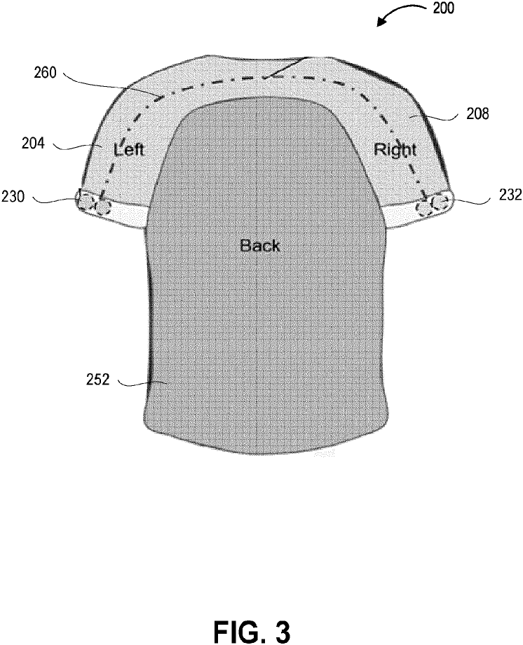

Reference is made to FIG. 3, which illustrates a rear view of the garment 200 of FIG. 2. The garment 200 includes a back portion 252. The garment 200 includes the first side section 204 and the second side section 208.

The garment 200 may include a conductive fiber 260 configured to electrically interconnect the sensor panel 230 associated with the first side section 204 and a sensor panel 232 associated with the second side section 208. The conductive fiber 250 may be an electrical pathway configured to interconnect one or more sensor panels and a controller device associated with the garment 200. The conductive fibre 260 may be knitted in the garment body. In some embodiments, the conductive fibre 260 may be integrated into or knitted into a garment seam.

In the example illustrated in FIG. 3, the conductive fiber 260 is knitted into the garment body across a yoke portion of the garment 200. In some embodiments, the conductive fibre 260 may be configured to conduct data signals and/or power signals.

In some embodiments, the sensor panel 230 associated with the first side section 204 and the sensor panel 232 associated with the second side section 208 may be configured as a complementary pair of bioelectrical impedance sensors for generating data for determining electrical impedance, in response to an electrical current transmitted through the user’s skin surface from the sensor panel 230 associated with the first side section 204 to the sensor panel 232 associated with the second side section 208, or vice versa.

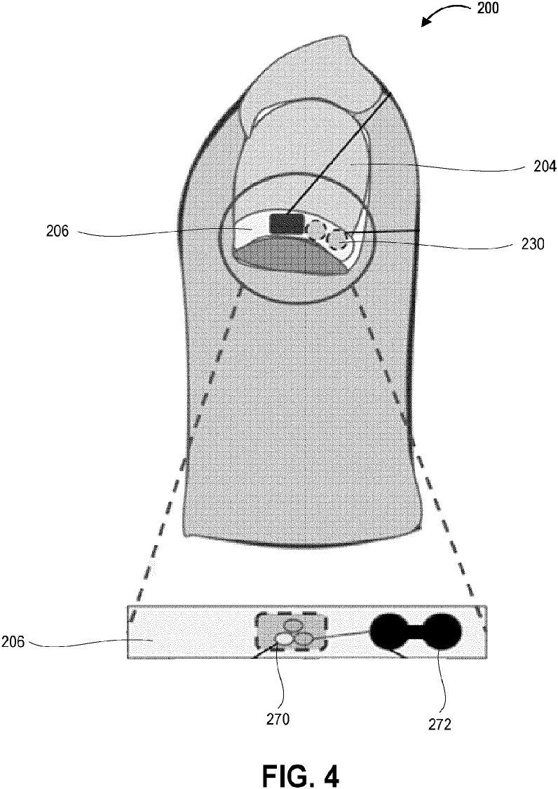

Reference is made to FIG. 4, which illustrates a side view of the garment 200 of FIG. 2. FIG. 4 also illustrates an enlarged view of the sensor panel 230 and/or the garment band 206 associated with the first side section 204. The enlarged view of the sensor panel and/or the garment band 206 is a partially transparent view of the garment band 206 for illustrating bio signal sensors, according to embodiments of the present application.

In FIG. 4, the sensor panel 230 includes one or more photoplethysmogram (PPG) sensors 270. The sensor panel 230 may include one or more electrocardiogram (ECG) sensors 272. Although PPG sensors 270 and ECG electrodes 272 are illustrated in FIG. 4, other sensors, such as ECG sensors, accelerometers, piezo sensors, may be contemplated.

As described, devices for physiological monitoring of a user may be provided in the garment 200. In some embodiments, the garment 200 may include a controller device (e.g., controller device 100 of FIG. 1), one or more sensor panels 230, and a network of conductive fibres for electrically interconnecting the controller device and the respective one or more sensor panels. In some embodiments, the controller device may conduct operations to monitor hemodynamic or blood pressure status of the garment user based on the one or more sensor panels affixed on a user facing side of the garment 200, where the one or more sensor panels may be positioned against a user limb (e.g., user arm) with substantially consistent pressure.

As an illustrative example, the controller device may conduct operations to determine hemodynamic data associated with the user based on bio signals generated by bio sensors of the sensor panels 230. The operations to determine hemodynamic data, such as blood pressure, may be based on pulse transit time (PTT) data received from the bio sensors. In some embodiments, the controller device may conduct operations to determine hemodynamic data based on a relationship or correlation between PTT data and blood pressure.

To illustrate, in some embodiments, the sensor panel 230 may include one or more bio sensors for measuring PTT data via central arteries of the garment user. PPT may be the time delay for a pressure wave to travel between two arterial positions. In some scenarios, PPT may be inversely related to blood pressure and may be estimated based on relative timing between proximal and distal waveforms indicative of an arterial pulse. Accordingly, in contrast to methods based on operating specialized devices such as a sphygmomanometer in combination with a stethoscope, the controller device may estimate blood pressure based on PTT data, where PTT data may be generated in a relatively non-invasive manner.

In some embodiments, the controller device may conduct operations to receive bio signals from one or more PPG sensors 270. The one or more PPG sensors 270 may generate bio signals based on optical transmittance or reflectance for generating bio signal waveforms indicative of proximal and distal blood volumes. As an illustrating example, a light-emitting diode (LED) may be paired with a photodetector (PD), and a small volume of user tissue (e.g., on a user limb) may be illuminated by the LED. Light transmitted through, or reflected back from, the user tissue may be detected by the photodetector. The detected light intensity may be reduced and may include dc and ac components. The dc components may indicate light absorption by nonpulsatile blood, skin, bone or other tissues. The ac component may represent light absorption by pulsatile arterial blood, including venous blood.

As an illustrative example, according to the Beer-Lambert-Bouguer relationship, as light of a given intensity (lo) may be incident on a volume, the transmitted light (I(t)) may be provided as:

$$1 \text{n} \left(\frac{I \left(t\right)}{{I}_{0}}\right) = - ∈ C V$$

where ε is an absorption coefficient, C is the concentration of the chromophore, and V is the volume of the medium. Accordingly, in the present example, the ac component of I(t) may be inversely related to the instantaneous arterial blood volume. The blood volume may be related to blood pressure via viscoelastic properties of an arterial wall. Accordingly, in some embodiments, the controller device described in the present application may conduct operations to estimate PTT based on bio signals generated by PPG sensors. In some embodiments, reflectance-mode PPG may be applicable to portions of the user’s body, such as the forehead, forearm, supraorbital artery, legs, or wrists.

In some embodiments, the controller device may conduct operations to receive bio signals from one or more ECG electrodes 272. The one or more ECG electrodes 272 may generate bio signals based on timing of cardiac electrical activity, which precedes the arterial pulse. In the present illustrating example, the time delay between the ECG waveform and a distal arterial waveform may be called the pulse arrival time (PAT). PAT may be equal to a sum of PTT and the preejection period (PEP). PEP may be determined by the ventricular electromechanical delay (VEMD) and isovolumic contraction period, which may be determined by ventricular and arterial pressures. For example, PEP may be expressed as:

$$\text{PEP =} \text{VEMD +} \left(\text{DP} - \text{VEDP}\right) / \text{dVICP}$$

where VEDP and dVICP may be the ventricular end-diastolic pressure and the average slope of ventricular isovolumic contraction pressure, respectively, and DP may be diastolic BP. In the present illustrating example, an ECG waveform may be used as a surrogate proximal waveform.

The above illustrating details associated with bio signals from one or more PPG sensors 270 or one or more ECG electrodes 272 for correlating PPT data or related waveforms and blood pressure are illustrating examples only, and the controller may conduct operations to determine blood pressure based on other methods or based on additional operations.

In some embodiments, one or more sensor panels affixed to a user facing side of a garment body to generate bio signals for determining hemodynamic data may include a pair of electrical bio impedance sensors for generating data associated with electrical conductivity of blood to generate or measure waveforms indicative of proximal and distal blood volumes. In some embodiments, electrical bioimpedance (EBI) or impedance cardiography (ICG) sensors may measure electrical blood conductivity, or a proximal waveform.

As an illustrating example when EBI or ICG sensors may be used, surface electrodes may be placed on a volume of tissue and a high-frequency electrical current may be injected into outer electrodes. A resultant differential voltage may be measured across inner electrodes and demodulated synchronously with an excitation frequency. As blood may be an electrical conductor, electrical current may travel through paths filled with blood. Thus, an ac component of the measured impedance (e.g., voltage divided by current) may represent pulsatile blood volume within tissue. In some embodiments, blood volume may be related to blood pressure via viscoelastic properties of the arterial wall. Accordingly, EBI or ICG sensors may be useful for PPT estimation.

In some embodiments, the one or more sensor panels affixed to a user facing side of a garment body to generate bio signals for determining hemodynamic data may include ballistocardiography (BCG) sensors. The BCG sensors may be configured to measure reactionary forces of the user body in response to cardiac ejection of blood into the aorta. In some embodiments, flexible strain or pressure sensors placed proximal to a superficial artery may measure waveforms indicative of or correlating to blood pressure.

In some embodiments, the garment 200 may include a combination of numerous bio sensor types for estimating blood pressure, or other physiological metrics / characteristics. As respective bio sensor types may be limited in ability to estimate physiological metrics of a user (e.g., there may be limits to correlation between PPT data and blood pressure for a given bio sensor type) or may be configured to estimate specific aspects of physiological metrics with a particular degree of accuracy, the controller device may conduct operations to estimate hemodynamic metrics based on bio signals received from a combination of bio sensor types, thereby estimating or determining hemodynamic metrics based on multi-modal bio signals.

In some embodiments, the garment 200 may include two or more bio sensors positioned at disparate portions of the garment user’s body, the controller device may conduct operations to estimate hemodynamic metrics (or other physiological metrics) based on bio sensor signals retrieved from disparate portions of the garment user’s body. In some embodiments, the controller device may conduct operations to determine physiological metrics based on a weighted calculation for estimating physiological metrics based on bio sensor signals from disparate portions of the garment user’s body. Accordingly, the garment 200 may include a sensor panel including at least two bio signal sensor types, and a controller device may estimate or determine hemodynamic metrics, or any other physiological metric, based on a combination of bio signal waveform data associated with each of the at least two bio signal sensor types.

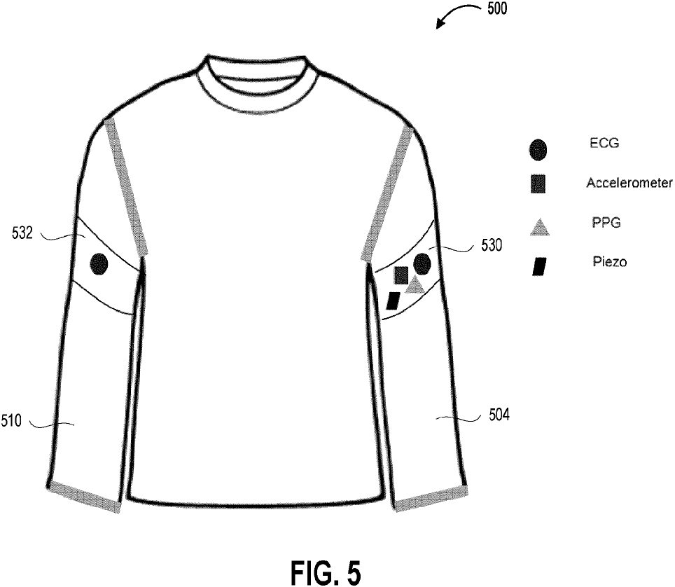

Reference is made to FIG. 5, which illustrates an elevation view of a garment 500, in accordance with an embodiment of the present application. The garment 500 may be a long sleeve shirt having a first sleeve 504 and a second sleeve 510.

In some embodiments, the first sleeve 504 may include a primary sensor panel 530 including a plurality of bio sensors types for generating bio signals. In the illustrated example, the primary sensor panel 530 may include one or more ECG sensors, one or more accelerometers, one or more PPG sensors, or one or more piezo sensors.

In some embodiments, the second sleeve 510 may include a complementary sensor panel 532. The complementary sensor panel 532 may include a different number and/or type of bio sensors. For example, the complementary sensor panel 532 may include one or more ECG sensors. The complementary sensor panel 532 may be positioned distal from the primary sensor panel 530. Further, the complementary sensor panel 532 may not mirror or include the same number and/or type of bio sensors as the primary sensor panel 530 and may generate a secondary set of bio signals.

The plurality of bio sensors of the garment 500 may be affixed to a user facing side of the garment 500. For ease of exposition, the primary sensor panel 530 and the complementary sensor panel 532 is illustrated as being translucent or partially transparent for illustrating the presence or positioning of the respective example bio signal sensors.

In some embodiments, one or more of the bio signal sensors may be configured to generate bio signals associated with cardiac, respiratory, olfactory, stretch, or hemodynamic parameters. In some embodiments, the generated bio signals may be for determining cardiac health, blood pressure, sleep metrics, fitness, wellness, or other relative measures of a garment user. In some embodiments, one or more bio signal sensors may be configured to periodically generate bio signals for estimation of physiological metrics including heart rate, arrhythmias including atrial fibrillation, blood pressure, user step count, calories, user activities, user sleep quality, or user sleep related breathing patterns. Other physiological metrics may be contemplated.

In some embodiments, the garment 500 may include one or more actuators for providing feedback to the garment user. In some embodiments, the one or more actuators may be haptic feedback elements, such as a servo motor, heating elements or pads, or other actuators for providing feedback to the garment user. In some embodiments, a controller device may be configured to activate one or more actuators in response to bio signals received from the one or more sensor panels. In some embodiments, the controller device may be configured to activate the one or more actuators in response to determined physiological data changes, such as blood pressure changes, that may be associated with a potential adverse health event. The controller device may activate the one or more actuators for providing feedback to the garment user on changing physiological conditions associated with the garment user.

In some embodiments, the garment 500 may include one or more accelerometers or piezo sensor integrated into the garment body for detecting user movement. In some embodiments, a controller device associated with the garment 500 may receive bio signals in response to receiving a trigger signal generated by at least one of the accelerometer or the piezo sensor indicating user movement.

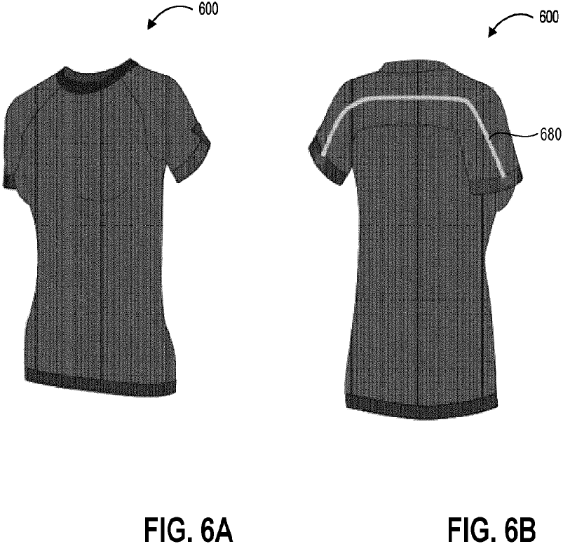

Reference is made to FIGS. 6A and 6B, which illustrate a front perspective view and a rear perspective view, respectively, of a garment 600 for detecting physiological data, in accordance with an embodiment of the present application.

The garment 600 may be an athletic t-shirt or may be a smart garment formed of a knitted textile. The smart garment may include a network of conductive and non-conductive fibres configured to transmit data and/or power signals. The smart garment may be configured to transmit data and/or power signals between a controller device and one or more sensor panels.

In some embodiments, the garment 600 may include a conductive strip 680 for electrically interconnecting sensor panels on opposing portions of the garment 600. For example, the conductive strip 680 may include one or more conducting fibres knitted into the garment 600 for electrically interconnecting sensor panels on opposing garment sleeves.

In the example illustrated in FIG. 6B, the conductive strip 680 may be configured to be routed along a contour of a shirt yoke. The shirt yoke may be a component of the garment 600 and may be a shaped pattern piece for forming a part of the garment that fits around the garment user’s neck and shoulders.

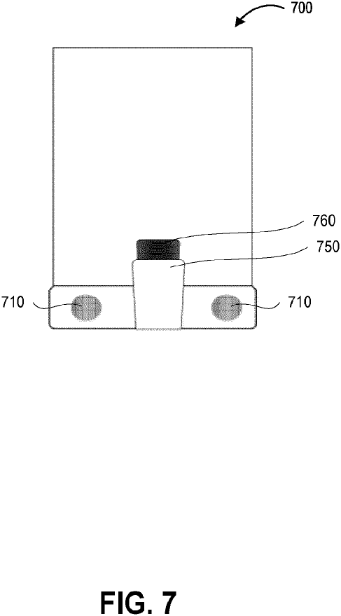

Reference is made to FIG. 7, which illustrates a garment sleeve 700, in accordance with an embodiment of the present application. The garment sleeve 700 may be configured to receive a user’s arm. The garment sleeve 700 may include one or more sensors 710 affixed to a user facing side of the garment sleeve 700. For ease of exposition, a portion of the garment sleeve 700 is illustrated as translucent or partially transparent so as to illustrate the position of the one or more sensors 710 on the garment sleeve 700.

In some embodiments, the garment sleeve 700 may include a textile enclosure 750 defining a cavity. The textile enclosure 750 may be knitted to the garment sleeve 700 and may project from a surface of the garment sleeve 700. The textile enclosure 750 may be configured to receive a controller device 760, and the textile enclosure 750 may be configured to electrically interconnect and/or mechanically interconnect the controller device 750 to the one or more sensors 710 or to the smart garment formed of a network of conductive and non-conductive fibres.

In some embodiments, the textile enclosure 750 may include a textile docking device received within the textile enclosure 750 and coupled to at least one conductive fibre of the textile substrate to electrically interconnect the received controller device 760 and the textile substrate.

In FIG. 7, the controller device 760 is illustrated as being coupled to the garment sleeve 700. It may be appreciated that the garment may include the textile enclosure 750 positioned at any other portion of the garment, and that the controller device 760 may be coupled to a portion of the garment other than the garment sleeve 700.

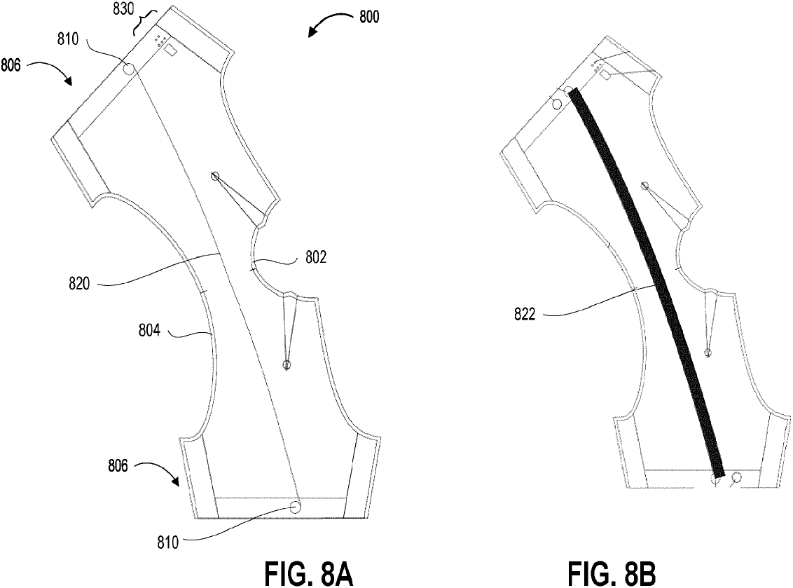

Reference is made to FIGS. 8A and 8B, which illustrate plan views of shirt yokes 800, in accordance with embodiments of the present application. The shirt yokes 800 may be components of a garment, such as a shirt, where the garment body may include a shaped pattern piece for forming the portion of the garment that fits around a user’s neck and shoulders. The shirt yoke 800 may include a neckline seam 802 and a back yoke seam 804. The shirt yoke 800 may include sleeve portions 806 that, when assembled, may form a left sleeve of a t-shirt garment and may form a right sleeve of a t-shirt garment.

FIG. 8A illustrates a shirt yoke 800 including one or more bio sensor 810 on a user facing side of a sleeve portion of the shirt yoke. The shirt yoke 800 may also include auxiliary interface components 830 including conductive pads configured to transmit electrical current to the garment user’s arm or light emitting diodes for providing visual indicators.

The shirt yoke 800 may include a conducting fibre 820 for electrically interconnecting the one or more bio sensors 810 positioned on the opposing garment sleeves.

FIG. 8B illustrates another embodiment of a shirt yoke similar to the shirt yoke 800 of FIG. 8A. In FIG. 8B, an inter-conducting path 822 may include a heat and/or pressure applied printed electronic tape. The inter-conducting path 822 may be configured to electrically interconnect one or more bio sensors 810 positioned on opposing garment sleeves. In some embodiments, the heat and/or pressure applied printed electronic tape may be configured to physically strengthen or protect the inter-conducting path 2822.

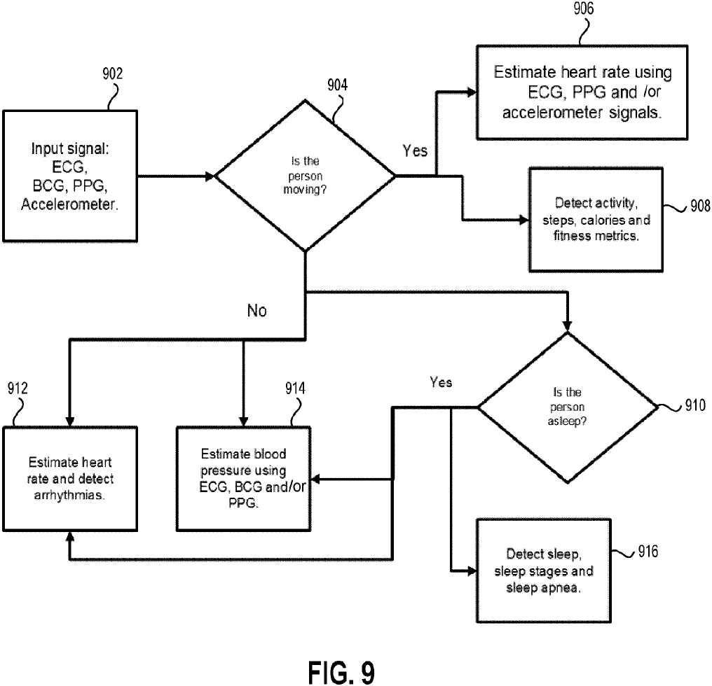

Reference is made to FIG. 9, which illustrates a flowchart of a method 900 of monitoring physiological conditions, in accordance with an embodiment of the present application. The method 900 may be conducted by the processor 102 of the example controller device 100 (FIG. 1). Processor readable instructions may be stored in the memory 106 and may be associated with the physiological monitoring application 112 or other processor readable applications not illustrated in FIG. 1. It may be appreciated that some examples described herein may refer to blood pressure or hemodynamic monitoring; however, other types of physiological monitoring may be contemplated.

At operation 902, the processor may receive, from a sensor panel, a primary set of bio signals. The primary set of bio signals may include signals based on at least one of ECG sensors, BCG sensors, PPG sensors, bio impedance sensors accelerometers, piezo sensors, or other types of sensors. In some embodiments, the processor may generate bio signal waveforms based on bio signal data received from at least one of ECG sensors, BCG sensors, PPG sensors, or bio impedance sensors. In some embodiments, the processor may generate user movement signals based on signal data received from at least one of accelerometer or piezo sensors.

At operation 904, the processor may determine whether the garment user is moving based on signal data received from the at least one of accelerometer or piezo sensors.

In the scenario that the processor determines that the garment user may be moving, at operation 906, the processor may estimate heart rate of the garment user based on bio signals received from at least one of the ECG sensors, PPG sensors, and/or accelerometer sensors.

In some embodiments, in the scenario that the processor determines that the garment user may be moving, the processor, at operation 908, may detect user activity (e.g., walking, running, exercising on an elliptical machine, swimming, etc.), user step count, user calorie burn count, and/or fitness metrics.

In the scenario that the processor determines that the garment user may not be substantially moving, the processor, at operation 912, may estimate heart rate and detect arrhythmias based on bio signal data received from at least one of the ECG sensors, BCG sensors, PPG sensors, or other bio signal sensor types. When the garment user may not be substantially moving, the garment user may be sitting, standing still, lying down, or in some other resting position.

In some embodiments, in the scenario that the processor determines that the garment user may not be substantially moving, the processor, at operation 914, may estimate blood pressure based on bio signals from at least one of ECG sensors, BCG sensors, and/or PPG sensors.

In some embodiments, the garment for detecting physiological data may include the combination of numerous bio sensor types for estimating blood pressure or other physiological metrics / characteristics. As respective bio sensor types may be limited in some aspects to estimate physiological metrics of a user (e.g., there may be accuracy limitations in correlating between PPT data and blood pressure for a given bio sensor type, or there may be accuracy limits in some environmental scenarios for one bio signal sensor type but not for another bio signal sensor type), the processor may conduct operations to estimate hemodynamic metrics based on bio signals received from a combination of bio sensor types. Accordingly, the processor may conduct operations to estimate or determine blood pressure based on multi-modal bio signals.

In the scenario that the processor determines that the garment user may not be substantially moving, the processor, at operation 910, may determine whether the garment user may be asleep. For example, the processor may determine whether the garment user may be substantially stationary for at least a threshold duration of time, thereby indicating that the user may be asleep. The processor may determine whether the garment user may have decreased heart rate for a prolonged period of time, thereby indicating that the user may be asleep.

In the scenario that the processor determines that the garment user may be asleep, the processor, at operation 914, may estimate blood pressure based on bio signals from at least one of ECG sensors, BCG sensors, and/or PPG sensors. In the present example, when the processor determines that the garment user may be asleep, the estimated blood pressure metrics may be associated with metadata indicating that the garment user was asleep. Accordingly, the controller device may store estimated blood pressure data associated with time durations when the garment user may be asleep and associated with time durations when the garment may be awake.

In the scenario that the processor determines that the garment user may be asleep, the processor, at operation 916, may detect user sleep stages and, in some embodiments, may detect the presence of sleep apnea. In some embodiments, the processor may conduct operations to detect user sleep stages based on heart rate data, bio electrical impedance data, or the like.

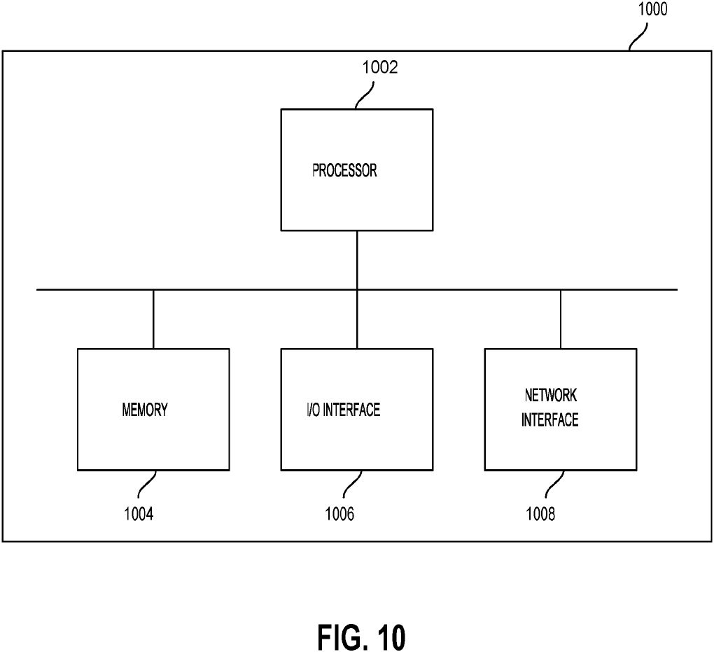

Reference is made to FIG. 10, which illustrates a block diagram of a computing device 1000, in accordance with an embodiment of the present application. As an example, the controller device 100 of FIG. 1 may be implemented using the example computing device 1000 of FIG. 10.

The computing device 1000 includes at least one processor 1002, memory 1004, at least one I/O interface 1006, and at least one network communication interface 1008.

The processor 1002 may be a microprocessor or microcontroller, a digital signal processing (DSP) processor, an integrated circuit, a field programmable gate array (FPGA), a reconfigurable processor, a programmable read-only memory (PROM), or combinations thereof.

The memory 1004 may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

The I/O interface 1006 may enable the computing device 1000 to interconnect with one or more input devices, such as a keyboard, mouse, camera, touch screen and a microphone, or with one or more output devices such as a display screen and a speaker.

In some embodiments, sensors of a smart garment described in the present application may interconnect with a data bus for shared communication or data messaging, which may be synchronized to a common clock element.

The networking interface 1008 may be configured to receive and transmit data sets, for example, to a target data storage or data structures. The target data storage or data structure may, in some embodiments, reside on a computing device or system such as a controller device.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

1-20. (canceled)

21. A smart garment for detecting physiological data, the smart garment comprising:
a garment configured to be worn on an upper body of a user;
a first garment portion having a primary sensor panel having an ECG sensor of a first sensor type;
a second garment portion having a complementary sensor panel, the complementary sensor panel including a complementary ECG sensor and a biosensor of a second sensor type different from the first sensor type;
one or more conductive fibres to electrically interconnect the primary sensor panel with the complementary sensor panel; and
a controller device configured to process a signal from the biosensor and ECG signals from the ECG sensors to detect a health event of the user.

22. The smart garment of claim 21, wherein the controller device is configured to use data collected by the ECG sensors and at least one of a BCG sensor and/or a PPG sensor to determine at least one of: pulse arrival time (PAT), the preejection period (PEP), ventricular electromechanical delay (VEMD), and/or pulse transit time (PTT).

23. The smart garment of claim 21, wherein the controller device is configured to determine blood pressure for the user based on one or more of determined PAT, PEP, VEMD, and/or PTT.

24. The smart garment of claim 21, further comprising an accelerometer for detecting user movement and/or position.

25. The smart garment of claim 24, wherein the primary sensor panel includes the accelerometer.

26. The smart garment of claim 24, wherein the controller device is configured to use accelerometer data to detect an adverse health event.

27. The smart garment of claim 21, wherein an enclosure is configured to mechanically interconnect the controller device to the garment.

28. The smart garment of claim 21, wherein the controller device detects arrhythmias based on signal data received from at least one of the ECG sensors or one or more BCG sensors.

29. The smart garment of claim 21, wherein the controller device is configured to determine that the user is asleep as a function of user heart rate.

30. The smart garment of claim 21, wherein the primary sensor panel and the complementary sensor panel are affixed to a user facing side of the garment.

31. The smart garment of claim 21, wherein biosensor is a ballistocardiogram (BCG) sensor.

32. The smart garment of claim 21, wherein the biosensor is a photoplethysmogram (PPG) sensor.

33. The smart garment of claim 21, wherein the controller device is configured to estimate hemodynamic metrics based on a combination of ECG sensor data and BCG sensor data.

34. The smart garment of claim 21, wherein the health event as an adverse health event is one of arrythmia, fainting, heart attack, and/or stroke.

35. The smart garment of claim 21, wherein the one or more conductive fibres are knit along with a base fabric in a layer of the garment.

36. The smart garment of claim 21, wherein the one or more conductive fibres are knitting into a seam of the garment.

37. The smart garment of claim 21, wherein the one or more conductive fibres form a length of interlocked fibers.

38. The smart garment of claim 21, wherein the one or more conductive fibres is/are knitted into the garment.

39. The smart garment of claim 21, further comprising an enclosure projecting from the garment for receiving the controller device, the enclosure configured to electrically interconnect the controller device and the one or more sensor panels using the one or more conductive fibres.

40. A smart garment for detecting physiological data, the smart garment comprising:
a garment configured to be worn on a body of a user;
a first garment portion having a primary sensor panel having an ECG sensor of a first sensor type;
a second garment portion having a complementary sensor panel, the complementary sensor panel including a complementary ECG sensor and a biosensor of a second sensor type different from the first sensor type;
one or more conductive fibres to electrically interconnect the primary sensor panel with the complementary sensor panel; and
a controller device configured to process a signal from the biosensor and ECG signals from the ECG sensors to detect a health event of the user.

41. A method for detecting physiological data using a smart garment, the method comprising the steps of:
receiving ECG signals from ECG sensors including:
an ECG sensor of a first sensor type of a first garment portion of a garment; and
a complementary ECG sensor of a complementary sensor panel of a second garment portion of the garment;
receiving a signal from a biosensor of a second sensor type different from the first sensor type, the biosensor also of the complementary sensor panel; and
processing the signal and the ECG signals to detect a health event of the user;
wherein one or more conductive fibres to electrically interconnect the primary sensor panel with the complementary sensor panel.
